(19) United States
(12) Patent Application Publication
(10) Pub. No.: US 2026/0345593 A1
(43) Pub. Date: Nov. 5, 2026

(54) ORAL FORMULATION OF N-PYRIDINYL ACETAMIDE DERIVATIVE FOR TREATING INTERSTITIAL LUNG DISEASE

(71) Applicant: AstraZeneca AB, Södertälje (SE)

(72) Inventors: Håkan Wikström, Gothenburg (SE); Caroline Ann Owen, Gaithersburg, MD (US); Susanne Prothon, Gothenburg (SE); Michael Lee Williams, Gothenburg (SE); Nicola Ferrari, Cambridge (GB); Veronika Sjöstrand, Gothenburg (SE); Rebecca Fransson, Gothenburg (SE); Alexandra Pavlovna Chukharkina, Gothenburg (SE)

(21) Appl. No.: 19/124,325

(22) PCT Filed: Nov. 3, 2023
(86) PCT No.: PCT/EP2023/080652

§ 371 Date: Apr. 25, 2025

Related U.S. Application Data
(60) Provisional application No. 63/382,311, filed on Nov. 4, 2022.

(51) Int. Cl. A61K 31/497; A61K 9/00; A61K 9/10; A61K 9/20; A61K 9/28; A61K 47/38

(52) U.S. Cl. CPC A61K 31/497; A61K 9/0053; A61K 9/10; A61K 9/2009; A61K 9/2013; A61K 9/2054; A61K 9/2813; A61K 9/2853; A61K 9/2866; A61K 47/38

(57) ABSTRACT

The present disclosure relates to oral formulations of an n-pyridinyl acetamide derivative, and the use of such formulations in the treatment of interstitial lung diseases.

The present specification claims benefit of priority to U.S. Provisional Application No. 63/382,311, filed 4 Nov. 2022, the content of which is hereby incorporated by reference in its entirety for all purposes.

The present disclosure relates to oral formulations of an n-pyridinyl acetamide derivative, and the use of such formulations in the treatment of interstitial lung diseases.

### BACKGROUND

Interstitial lung diseases are a group of rare diffuse parenchymal lung disorders associated with substantial morbidity and mortality that share common clinical and pathophysiological features, but also demonstrate a diverse etiology, and prognosis (Olsen et al 2018). ILDs affect the interstitium or the connective tissue stroma that separates the epithelial and endothelial barriers in the lungs. ILDs are divided into 5 broad clinical categories: ILDs related to distinct primary diseases (eg, sarcoidosis), ILDs related to environmental exposures (eg, hypersensitivity pneumonitis), ILDs induced by drugs or irradiation, ILDs associated with connective tissue diseases (eg, scleroderma and rheumatoid arthritis), and idiopathic interstitial pneumonias (eg, idiopathic pulmonary fibrosis, IPF). Although ILDs are heterogeneous, they have common genetic risk factors and shared downstream pathways leading to fibrogenesis.

Among the ILD-PF, IPF is the most common and lethal. The incidence of IPF has risen over time and a conservative estimate of the incidence in Europe and North America is 3 to 9 cases per 100000 people per year (Hutchinson 2015). IPF generally presents in individuals over the age of 50 years and is more common in men than women (Richeldi 2017). Smoking cigarettes is a known risk factor (Baumgartner 1997). IPF is characterized by the deposition of excessive amounts of extracellular matrix proteins in the lungs, which replaces the normal architecture of the distal lung and impairs gas exchange leading to progressive exertional dyspnea and decline in lung function, and ultimately to respiratory failure and death (Richeldi 2017).

Idiopathic pulmonary fibrosis is characterized pathologically by the pattern of usual interstitial pneumonitis and the presence of pathognomonic clusters of activated (myo)fibroblasts (fibroblastic foci). Although the etiology of IPF is still unclear, it is thought to be due to repetitive epithelial injury in genetically susceptible individuals inducing an aberrant wound healing response. This is characterized by excessive and abnormal epithelial repair, (myo)fibroblast activation driven by increased production of pro-fibrotic growth factors such as TGF-β, epithelial-to-mesenchymal transition, interstitial collagen deposition by (myo)fibroblasts, and immune cell dysfunction including the accumulation of pro-fibrotic macrophages in the lung (Richeldi 2017). Although the course of the disease can be variable, the fibrotic response in the lungs of IPF patients is often relentlessly progressive leading to progressive impairment of gas exchange, respiratory failure and death. In the absence of anti-fibrotic therapies, the median survival from the time of diagnosis is ~2 to 4 years (Ley 2011).

A proportion of patients with ILDs other than IPF also develop a progressive fibrosing phenotype associated with decline in lung function, worsening symptoms and QoL, and early mortality. These include idiopathic nonspecific interstitial pneumonia, ILDs associated with autoimmune diseases such as rheumatoid arthritis-associated interstitial lung disease (RA-ILD) and systemic sclerosis-associated interstitial lung disease (SSc-ILD), chronic hypersensitivity pneumonitis, chronic sarcoidosis, exposure-related ILDs such as silicosis, and unclassifiable idiopathic interstitial pneumonia (Wollin 2019). The pathogenesis and clinical features of other ILD-PF is similar to that of IPF.

Currently there are only 2 therapies that have been approved to treat patients with IPF: pirfenidone and nintedanib. Both of these therapies have anti-fibrotic activities and slow the rate of decline in lung function (King 2014 and Richeldi 2014). However, both therapies are associated with troublesome GI side effects and many patients with IPF are not able to tolerate these medications. Nintedanib is also approved to treat patients with ILD-PF other than IPF and has been shown to slow the rate of decline in lung function in these patients (Wells 2020). Tocilizumab (a monoclonal antibody directed against the IL-6 receptor) has been shown to slow progression of lung disease in the subset of ILD patients with SSc-ILD (Khanna 2020) and has recently been approved to treat this disease. However, none of the approved anti-fibrotic therapies relieves symptoms, halts lung function decline, or restores normal lifespan in patients with IPF or other ILD-PF. Thus, there is a pressing unmet need for therapies that are more effective and better tolerated for patients with IPF and other ILD-PF.

WNT ligands are glycoproteins that are secreted by various cells and bind to extracellular receptors including WNT ligands, the ligands then bind to receptors including the 10 members of the Frizzled receptor family (FZD) and their co-receptors (LRP5/6). Binding of WNT ligands to FZD and LRP5/6 can trigger either canonical or non-canonical WNT signalling (Baarsma and Königshoff 2017).

The LEF/TCF family triggers downstream expression of pro-fibrotic genes in multiple cellular culprits involved in the pathogenesis of IPF including alveolar epithelial cells, (myo)fibroblasts, and alveolar macrophages. TGF-β can activate canonical WNT signalling in lung and skin fibroblasts, with nuclear translocation of β-catenin and increased transcription of WNT target genes. Further activation of WNT signalling by TGF-β occurs via downregulation of endogenous WNT antagonists (Distler 2019). Non-canonical WNT signalling follows binding of specific WNTs (eg, WNT 4 and 5a) to FZD receptors, but is independent of LRP5/6 co-receptors. Non-canonical WNT signalling results in activation of intracellular signalling molecules involved in planar cell polarity, calcium/calmodulin-dependent protein kinase II and other less well-defined pathways (Baarsma and Königshoff 2017). However, WNT signalling is complex and an individual WNT scan can activate both the canonical and non-canonical pathways.

Enhanced WNT signalling contributes to the pathogenesis of IPF; antibody mediated inhibition of WISP-1 reduces pulmonary fibrosis in bleomycin-treated mice by altering alveolar epithelial cell function and reprogramming and reducing fibroblast activation (Königshoff 2009). Unbiased gene expression profiling in IPF lung tissue identified a WNT signature (Königshoff 2008). In addition, canonical WNT signalling is elevated in various cells in the lungs of IPF patients and in the lungs of experimental animals with pulmonary fibrosis. The expression of WNT1, 7B, 10B, FZD2, FDZ3, and β-catenin, LEF1, and WISP-1 genes is elevated in the lungs of patients with IPF versus healthy controls (Königshoff 2008). Increased staining for WNT1 and 3A has been detected in bronchial and alveolar epithelial cells and increased nuclear staining for β-catenin has been detected in bronchiolar epithelial cells and fibroblasts in fibroblastic foci (Königshoff 2008, Chilosi 2003). Furthermore, increased WNT10A expression in the lungs has been related to poorer survival rates in patients with IPF (Oda 2016).

Additional support for canonical WNT signalling in the pathogenesis of IPF are studies showing that the expression of LRP-5 and -6 are elevated in IPF lungs and that LRP5 transcript levels correlate with IPF progression and negatively with the diffusing capacity of the lung for carbon monoxide. In addition, mice genetically deficient in LRP5 were protected from bleomycin-induced pulmonary fibrosis (Lam 2014).

Non-canonical WNT signalling also contributes to the pathogenesis of IPF. Transforming growth factor-ß has been shown to drive secretion of non-canonical WNTs. The expression of WNT5A, a non-canonical WNT ligand, is increased in fibroblasts in patients with IPF and is strongly regulated by TGF-β (Distler 2019). WNT5B signals via FZD8 in a β-catenin independent fashion. There is high-level expression of FZD8 in IPF lungs and expression levels correlate with disease progression (Lam 2014). Genetic silencing of FZD8 reduces pro-fibrotic signalling in fibroblasts in vitro and bleomycin-mediated pulmonary fibrosis in mice (Spanjer 2016)

Increased WNT signalling has also been linked to the pathogenesis of other ILD-PF (Adegunsoye 2019; Distler 2019). Preliminary gene expression studies show that WNT gene expression is elevated in lung samples from patients with other ILD-PF. Microarray analyses of human tissue samples from patients with systemic sclerosis demonstrate alterations in the expression of a number of β-catenin target genes (matrix metalloproteinase-7), osteopontin, cyclin D1, secreted FZD-related protein2, peroxisome proliferator-activated receptor δ, and WISP-1) and pathway components (WNT2b, WNT5b, and WNT inhibitory factor 1). Patients with SSc-associated advanced pulmonary fibrosis have increased nuclear β-catenin accumulation in fibroblastic foci in their lungs (Lam 2011).

Forced activation of β-catenin signalling in 3 independently derived sources of normal human lung fibroblasts promotes proliferation and migration indicating that activation of β-catenin signalling in pulmonary fibroblasts may be a common feature of lung fibrosis, contributing to fibroproliferative and migratory activities of (myo)fibroblasts associated with the disease. WNT5a protein levels were elevated in plasma samples from RA-ILD patients compared with non-RA-ILD patients, and levels positively correlated with the plasma level of rheumatoid factor (Yu 2019). Higher plasma levels of WNT5A were also found in patients with UIP than those with nonspecific interstitial pneumonia and other ILD patterns. RA-ILD disease severity was correlated with the circulating WNT5a levels as assessed by high-resolution computed tomography-UIP scores (Yu 2019).

Three porcupine inhibitors (CGX1321: NCT03507998; ETC-159: NCT02521844; WNT974: NCT01351103) are currently being evaluated in Phase I clinical studies of patients with advanced or metastatic cancer (Shah 2021). To date there have been no clinical studies evaluating PORCN inhibitors in patients with IPF or other ILD-PF. However, in experimental fibrosis models, treatment with different PORCN inhibitors significantly reduced fibrosis in the kidney in a renal fibrosis model (Madan 2016) and in the skin and lungs in 2 murine models of scleroderma (Chen 2017).

### SUMMARY

In some embodiments, disclosed is a method of treating interstitial lung disease in a subject in need thereof, comprising orally administering to the subject AZD5055 in a dose of between 5 and 35 mg per day.

This dose may be delivered in a single dosage unit, or in multiple dosage units.

In some embodiments, disclosed is an oral dosage unit which comprises AZD5055 in an amount of between 2.5 and 35 mg.

### DETAILED DESCRIPTION

The term AZD5055 refers to a compound with the chemical name 2-[4-(2-methyl-4-pyridyl)pyrrolo[3,2-c]pyridin-1-yl]-N-(5-pyrazin-2-yl-2-pyridyl)acetamide and structure shown below:

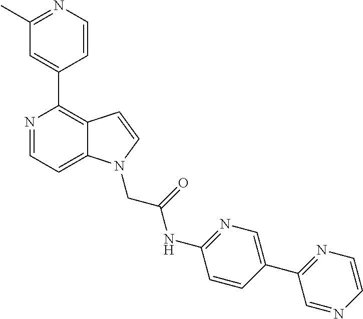 [SMILES: Cc1cc(-c2nccc3c2ccn3CC(=O)Nc2ccc(-c3cnccn3)cn2)ccn1]

AZD5055, also known as RXC006, is a potent, and selective inhibitor of the intracellular enzyme, porcupine (PORCN). Porcupine (PORCN) is a membrane-bound O-acyltransferase required for and dedicated to palmitoylation of all Wnt ligands, an essential step in the processing of Wnt ligands for secretion (Herr 2012).

WNT signalling makes critical contributions to the pathogenesis of ILD-PF via activities on multiple key cell types implicated in the pathogenesis of these diseases. AZD5055 is expected to inhibit WNT signalling in epithelial cells, fibroblasts, and alveolar macrophages in the lungs of patients with IPF and other ILD-PF and thereby limit the progression of pulmonary fibrosis and improve quality of life and survival in patients with these diseases. Preliminary studies of AZD5055 show that it reduces bleomycin-mediated fibrosis in the lungs of rats, carbon tetrachloride-induced hepatic fibrosis in mice, and renal fibrosis in a UUO model in mice (Bunyard 2019).

The synthesis of AZD5055 is described in WO2016/055790 (compound ID No. 23), the contents of which are hereby incorporated by reference in their entirety. In some embodiments, a free base AZD5055 is administered to a subject. In some embodiments, crystalline AZD5055 is administered to a subject.

The term “proton pump inhibitor” refers to a class of medications that cause a profound and prolonged reduction of stomach acid production. They do so by irreversibly inhibiting the stomach's H⁺/K⁺ ATPase proton pump. Examples of proton pump inhibitors include: Omeprazole; Lansoprazole; Dexlansoprazole; Esomeprazole; Pantoprazole; Rabeprazole; and Ilaprazole.

The term “rabeprazole” refers to a compound with the chemical name (RS)-2-([4-(3-methoxypropoxy)-3-methylpyridin-2-yl]methylsulfinyl)-1H-benzo[d]imidazole and structure shown below:

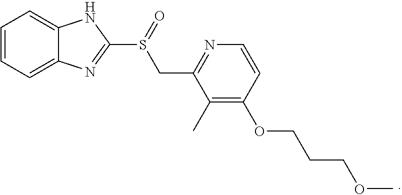

The term “nintedanib” refers to a compound with the chemical name methyl (3Z)-3-{[(4-{methyl[(4-methylpiperazin-1-yl)acetyl]amino}phenyl)amino](phenyl)methylidene}-2-oxo-2,3-dihydro-1H-indole-6-carboxylate and structure shown below:

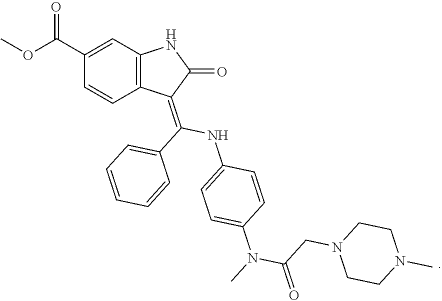

The language “treat,” “treating” and “treatment” includes the reduction or inhibition of enzyme or protein activity related to PORCN or WNT in a subject, amelioration of one or more symptoms of interstitial lung diseases, such as idiopathic pulmonary fibrosis in a subject, or the slowing or delaying of progression of interstitial lung diseases, such as idiopathic pulmonary fibrosis in a subject. The language “treat,” “treating” and “treatment” also includes the reduction or inhibition of the growth of clusters of activated (myo)fibroblasts in a subject.

The language “inhibit”, “inhibition” or “inhibiting” includes a decrease in the baseline activity of a biological activity or process.

The term “subject” includes warm-blooded mammals, for example, primates, dogs, cats, rabbits, rats, and mice. In some embodiments, the subject is a primate, for example, a human. In some embodiments, the subject is suffering from interstitial lung diseases, such as idiopathic pulmonary fibrosis.

In some embodiments, disclosed is a method of treating interstitial lung disease in a subject in need thereof, comprising orally administering to the subject AZD5055 in a dose of between 5 and 35 mg per day.

Further disclosed is AZD5055 for use in the treatment of an interstitial lung disease, the method comprising the oral administration of AZD5055 in a dose of between 5 and 35 mg per day. Additionally disclosed is the use of AZD5055 in the manufacture of a medicament for the treatment of interstitial lung disease by oral administration of a dose of between 5 and 35 mg per day.

In further embodiments, the dose per day is between 10 and 30 mg per day, or 15 and 25 mg per day.

In some embodiments, AZD5055 is administered in a dose of 5 mg per day, about 10 mg per 30 day, about 15 mg per day, about 20 mg per day, about 25 mg per day, about 30 mg per day or 35 mg per day. In some embodiments, AZD5055 is administered in a dose of about 5 mg per day, about 10 mg per day, about 15 mg per day, about 20 mg per day, about 25 mg per day, about 30 mg per day or about 35 mg per day. In some embodiments, AZD5055 is administered in a dose of about 5 mg per day. In some embodiments, AZD5055 is administered in a dose of about 15 mg per day.

The dose per day may be 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg or 35 mg.

In some embodiments, AZD5055 is administered once a day (0D). In some embodiments, AZD5055 is administered in a dose of 5 mg QD, about 10 mg QD, about 15 mg 0D, about 20 mg QD, about 25 mg QD, about 30 mg QD or 35 mg QD. In some embodiments, AZD5055 is administered in a dose of about 5 mg QD, about 10 mg QD, about 15 mg QD, about 20 mg QD, about 25 mg QD, about 30 mg 0D or 35 mg QD. In some embodiments, AZD5055 is administered in a dose of about 5 mg QD. In some embodiments, AZD5055 is administered in a dose of about 15 mg QD.

In some embodiments, AZD5055 is administered twice a day (BID). In some embodiments, AZD5055 is administered in a dose of 2.5 mg BID, about 5 mg BID, about 7.5 mg BID, about 10 mg BID, about 12.5 mg BID, about 15 mg BID or 17.5 mg BID. In some embodiments, AZD5055 is administered in a dose of about 2.5 mg BID, about 5 mg BID, about 7.5 mg BID, about 10 mg BID, about 12.5 mg BID, about 15 mg BID or about 17.5 mg BID.

In some embodiments, AZD5055 is continuously administered in the treatment cycle.

The term “continuous” or “continuously” refers to administration of a therapeutic agent, e.g. AZD5055, at regular intervals without stopping or interruption, i.e., no void day. By “void day”, it is meant a day when a therapeutic agent is not administered.

A “cycle”, “treatment cycle” or “dosing schedule”, as used herein, refers to a period of combination treatment that is repeated on a regular schedule. For example, the treatment can be given for one week, two weeks, or three weeks wherein AZD5055 is administered. In some embodiments, a treatment cycle is about 1 week to about 3 months. In some embodiments, a treatment cycle is about 5 days to about 1 month. In some embodiments, a treatment cycle is about 1 week to about 3 weeks. In some embodiments, a treatment cycle is about 1 week, about 10 days, about 2 weeks, about 3 weeks, about 4 weeks, about 2 months, or about 3 months. In some embodiments, the treatment cycle is for 1 year, or more.

The daily dose of between 5 and 35 mg per day is considerably lower than the originally predicted human therapeutic dose based on the preclinical data on AZD5055 which was 164 mg (given as 83 mg BID). This originally predicted therapeutic dose is based on the in vitro IC₅₀ value of 0.64 nmol/L (269.7 ng/L or 0.0003 mg/L), generated in a β-catenin reporter assay using rodent cells (Bunyard 2019). A coverage of 3× in vitro IC₅₀ over 24 hours in vivo was chosen to achieve target engagement and efficacy as it was expected to result in continuous inhibition of PORCN>75% in magnitude across the dosing interval. A coverage of 3× in vitro IC₅₀ over 24 hours in vivo was expected to be efficacious based on the exposures that reduced pulmonary fibrosis in the rat and mouse bleomycin in vivo studies (Bunyard 2019). The 3× in vitro IC₅₀ approach was also supported by clinical data published on Wnt974, a PORCN inhibitor currently in clinical development for the treatment of subjects with advanced cancers (Rodon 2021). The predicted therapeutic exposures of AZD5055 would have provided coverage of the target and a similar free C_{trough}, as the Wnt974 10 mg once daily clinical dose. The Wnt974 10 mg once daily clinical dose resulted in approximately 90% reduction in Axin2 (a Wnt pathway biomarker) expression levels in skin samples from subjects with advanced cancers. In subjects receiving this dose, the average C_{trough} at steady state corresponds to 3.7×IC₅₀. Human PK parameters were predicted from in vitro and nonclinical in vivo studies. The total plasma clearance was predicted to be 9.1 mL/min/kg, the volume of distribution under steady state conditions 2.4 L/kg, and the t_{1/2}, 3 hours. The bioavailability and the absorption rate constant (ka) were predicted to be 22%, and 1.0 h-1, respectively. Using these PK parameters the plasma steady state exposure (total concentrations) was estimated at a Cₘᵢₙ (minimum plasma concentration) of 0.023 μmol/L (0.010 mg/L), a Cₘₐₓ (maximum plasma concentration of 0.18 μmol/L (0.076 mg/L), and an AUC₍₀₋₂₄₎ (area under the plasma concentration-time curve from time 0 to 24 hours) of 2.24 μmol·h/L (0.944 mg·h/L) at the originally predicted therapeutic twice daily dose of 83 mg.

In some embodiments, AZD5055 is taken on an empty stomach, with no food two hours before, and one hour after. In other embodiments, AZD5055 is taken with food.

Taking AZD5055 with or without food will affect the Cₘₐₓ, AUC_{inf} (Area under the plasma concentration-time curve from zero to infinity), and AUCₗₐₛₜ (area under the plasma concentration-time curve from zero to the time of the last quantifiable concentration). Taking AZD5055 with food may increase the AUC_{inf} and AUCₗₐₛₜ.

In some embodiments, a method of treating interstitial lung disease in a subject in need thereof additionally comprises the separate, sequential or simultaneous administration of a proton pump inhibitor or a pharmaceutically acceptable salt thereof.

Further disclosed is AZD5055 for use in the treatment of an interstitial lung disease, the method additionally comprising the separate, sequential or simultaneous administration of a proton pump inhibitor or a pharmaceutically acceptable salt thereof. Additionally disclosed is the use of AZD5055 in the manufacture of a medicament for the treatment of interstitial lung disease the treatment additionally comprising the separate, sequential or simultaneous administration of a proton pump inhibitor or a pharmaceutically acceptable salt thereof.

Dosing AZD5055 alongside a proton pump inhibitor is expected to be important in patients suffering from conditions where excess stomach acid is produced, including dyspepsia, peptic ulcer disease, and gastroesophageal reflux disease. Proton pump inhibitors reduce the acidity of the stomach contents, whilst the solubility of AZD5055 is known to be pH dependent. Therefore, such a combination may have been expected to require a higher daily dose of AZD5055 and/or require moving to a twice daily dosing from once daily dosing.

In some of these embodiments, the proton pump inhibitor is rabeprazole. In some embodiments, rabeprazole is administered orally. In some embodiments, rabeprazole is in tablet dosage form, more particularly delayed-release tablet dosage form. In some embodiments, rabeprazole is administered in a dose of about 10 or 20 mg orally once a day (QD) or 10 or 20 mg orally twice a day (BID). In some embodiments, the dose comprises one 10 mg tablet, two 10 mg tablets or one 20 mg tablet.

In some embodiments, AZD5055 and rabeprazole are taken together on an empty stomach, with no food two hours before, and one hour after. In other embodiments, rabeprazole is taken on an empty stomach, with no food two hours before, and one hour after, and AZD5055 is taken with food.

In some embodiments, disclosed is an oral dosage unit which comprises AZD5055 in an amount of between 2.5 and 35 mg.

In some embodiments, the oral dosage unit comprises the daily dose of AZD5055. In other embodiment, the oral dosage unit comprises half the daily dose of AZD5055.

In some embodiments, the oral dosage unit is an aqueous suspension.

The suspension may comprise additional excipients include viscosity agents (for example carboxymethylcellulose sodium) sweeteners and colorants. In some embodiments, the viscosity agent is carboxymethylcellulose sodium and is present in an amount of 5 mg per mL of suspension.

In some embodiments, the oral dosage unit is a tablet.

In some of these embodiments, the tablet comprising one or more pharmaceutical excipients selected from the group consisting of: diluents, compression aids, disintegrants, and glidants/lubricants.

In some embodiments, the diluent is a sugar or sugar-containing, except mannitol. For example, the diluent may be lactose, dextrin, glucose, sucrose, sorbitol. In some embodiments, the diluent is microcrystalline cellulose. It has been found from a compatibility study that the commonly used diluent, mannitol, is not compatible with AZD5055. When used as a diluent, additional amounts of degradation products were identified.

In some embodiments, the diluent is an inorganic compound, such as silicates, calcium and magnesium salts, sodium or potassium chloride.

In some embodiments, the compression aid (which may also be termed a binder or granulating agent) is selected from microcrystalline cellulose (MCC), xylitol, di-calcium phosphate, lactose monohydrate and starch. In some embodiments, the compression aid is di-calcium phosphate (a.k.a. dibasic calcium phosphate).

In some embodiments, the compression aid is between 10 and 30 wt % of the tablet core. In some of these embodiments, it is between 15 and 25 wt % of the tablet core. In some embodiments it is 22 wt % of the tablet core.

In some embodiments, the lubricant/glidant is glyceryl behenate. It has been found from a compatibility study that the commonly used glidant, silicon dioxide, and lubricant, magnesium stearate, are not compatible with AZD5055. When these were used as a glidant and lubricant respectively, additional amounts of degradation products were identified.

In some embodiments, the lubricant/glidant is between 2 and 15 wt % of the tablet core. In some of these embodiments, it is between 5 and 12 wt % of the tablet core. In some embodiments it is 10 wt % of the tablet core.

In some embodiments, the disintegrant is a compound which swells or dissolves in water, such as starch, cellulose derivatives, alginates, and crospovidone. In some embodiments, the disintegrant is croscarmellose sodium.

In some embodiments, the disintegrant is between 2 and 15 wt % of the tablet core. In some of these embodiments, it is between 5 and 12 wt % of the tablet core. In some embodiments it is 10 wt % of the tablet core.

In some embodiments, the tablet does not contain mannitol, silicon dioxide or magnesium stearate.

In some embodiments, the tablet is coated. The coating may comprise one or more excipients such as a film former, plasticizer, opacifier and colourants.

In other embodiments, the tablet is uncoated.

In some embodiments, disclosed is a kit comprising: a first pharmaceutical composition comprising AZD5055; a second pharmaceutical composition comprising a proton pump inhibitor or a pharmaceutically acceptable salt thereof; and instructions for using the first and second pharmaceutical compositions in combination.

Interstitial lung diseases (ILDs) include ILDs related to distinct primary diseases (eg, sarcoidosis), ILDs related to environmental exposures (eg, hypersensitivity pneumonitis), ILDs induced by drugs or irradiation, ILDs associated with connective tissue diseases (eg, scleroderma and rheumatoid arthritis), and idiopathic interstitial pneumonias (eg, IPF).

Sarcoidosis is a systemic disease characterised by the formation of immune granulomas in organs, especially in the lungs and lymphatic system. It is estimated that up to 20% of patients diagnosed with sarcoidosis develop fibrotic lung disease, with pulmonary fibrosis presenting as dyspnoea, cough and hypoxaemia.

ILDs related to environmental exposures include hypersensitivity pneumonitis, asbestosis and silicosis. Hypersensitivity pneumonitis (HP) is an immunologically initiated lung disease triggered by the repeated inhalation of a large variety of environmental organic antigens and/or chemicals, to which the genetically susceptible subject has been previously sensitised. Asbestosis is caused by exposure to asbestos. Silicosis is caused by exposure to free crystalline silicon dioxide or silica.

There is a spectrum of connective tissue diseases (CTDs) characterised by an underlying mechanism of systemic autoimmunity and immune-mediated organ damage, which may develop pulmonary complications throughout the disease course. These CTDs include rheumatoid arthritis (RA), scleroderma (systemic sclerosis (SSc)), idiopathic inflammatory myopathy (polymyositis and dermatomyositis), Sjögren's syndrome, systemic lupus erythematosus and mixed CTDs. RA and SSc are most commonly associated with progressive-fibrosing ILD.

Idiopathic pulmonary fibrosis (IPF) is characterized by the deposition of excessive amounts of extracellular matrix proteins in the lungs, which replaces the normal architecture of the distal lung and impairs gas exchange leading to progressive exertional dyspnea and decline in lung function, and ultimately to respiratory failure and death.

In some embodiments, treatment with nintedanib may be administered concurrently. Such additional treatments may be dosed at the current standard of care or at a reduced dose.

In some embodiments, the subject may have previously been treated with nintedanib.

### EXAMPLES

The compounds of the application will now be further explained by reference to the following non-limiting examples.

### Suspension Formulation

| Component | Quantity (per mL) | | Function |
|---|---|---|---|
| AZD5055 | 1 to 20 | mg | Active drug substance |
| Carboxymethylcellulose | 5 | mg | Viscosity agent |
| sodium | | | |

| | | |
|---|---|---|
| Purified water | To 1 mL | Solvent |

### Tablet Formulation

| Component | Amount - Core | | Function |
|---|---|---|---|
| AZD5055 | 5 mg to 35 mg | | Active drug substance |
| Dibasic calcium phosphate | 30.8 | mg | Compression Aid |
| anhydrous | | | |
| Croscarmellose sodium | 7 | mg | Disintegrant |
| Glyceryl behenate | 7 | mg | Lubricant |

| | | |
|---|---|---|
| Microcrystalline cellulose | To 140 mg | Diluent |

The tablet is formed as a 7 mm round, biconvex tablet by roller compression, and then is coated with about 4.3 wt % of a coating pre-mix to give a coverage of about 5 mg/cm². The coating pre-mix is Aquarius™ Prime BAP312542 Brown and consists of HPMC (film former), PEG (plasticizer), TiO₂ (opacifier) and FeOₓ (colourants).

### Example 1. First Clinical Study to Investigate Safety, Tolerability and Pharmacokinetics

This study is in two parts—part 1 involves testing a single ascending dose, whilst part 2 involves testing multiple ascending doses.

Part 1 comprises a Screening Period of a maximum of 6 weeks. This is followed by a Treatment Period during which subjects are resident at the Clinical Unit from 1 day before investigational medicinal product (IMP) administration (Day −1) until at least 72 hours after IMP administration (Day 4). Subjects receive a single oral dose of AZD5055 or placebo on Day 1. This is followed by a Follow-up Visit within 6±1 day after the IMP dose.

Part 2 comprises a Screening Period of a maximum of 6 weeks. This is followed by a treatment period during which subjects are resident at the Clinical Unit from 1 day before IMP administration (Day −1) until at least 72 hours after the last dose given on Day 16 (Day 19). Subjects are dosed for a total of 15 days and receive a single once daily morning dose of AZD5055 or placebo on Day 1 and on Days 3 through Day 16, with no dosing on Day 2. This is followed up a Follow-up Visit within 6±1 day after the IMP dose, and an additional Follow-up Visit within 29±2 days after the last IMP dose.

For cohorts with QD dosing regimens, the Screening Period is followed by a Treatment Period during which subjects are resident at the Clinical Unit from 1 day before IMP administration (Day −1) until at least 72 hours after the last dose given on Day 16 (Day 19). Subjects are dosed for a total of 15 days, receiving a single QD morning dose of AZD5055 or placebo on Day 1 and on Days 3 through Day 16.

For cohorts with BID dosing regimens, the Screening Period is followed by a Treatment Period during which subjects are resident at the Clinical Unit from 1 day before IMP administration (Day −1) until at least 72 hours after the last dose given on Day 16 (Day 19).

Subjects are dosed for a total of 15 days, receiving a single morning dose of AZD5055 or placebo on Day 1 and Day 16, and repeated BID dosing on Day 3 through Day 15, 12 hours (±30 minutes) apart.

For both cohorts, the Treatment Period is followed by a Follow-up Visit within 6±1 day after the last IMP dose, and an additional Follow-up Visit within 29±2 days after the last IMP dose.

### Study Objective

### Primary Objective(s)

Part 1: SAD (Single Ascending Dose)—to assess the safety and tolerability of AZD5055 following oral administration of single ascending doses to healthy subjects.
Part 2: MAD (Multiple Ascending Dose)—to assess the safety and tolerability of AZD5055 following oral administration of multiple ascending doses to healthy subjects.

### Secondary Objective(s)

Part 1: SAD—To characterize the PK of AZD5055 following oral administration of single ascending doses to healthy subjects.
Part 2: MAD—To characterize the PK of AZD5055 following oral administration of multiple ascending doses to healthy subjects.

### Exploratory Objective(s)

### Part 1: SAD

To evaluate target engagement in hair follicles (Cohort 1 and 2).
To evaluate target engagement in whole blood.
To assess the effect of AZD5055 on biomarkers of bone turnover.
To evaluate cardiac safety as assessed by 12-lead safety electrocardiograms (ECGs) and dECGs, including the intention of obtaining a TQT substitute.
To evaluate changes in a panel of renal biomarkers in urine if indicated by the 3-month preclinical toxicity study.
To investigate the presence and measure levels of metabolites of AZD5055 in plasma and urine. The results will be used to guide future investigations of exposure to metabolites of AZD5055.

### Part 2: MAD

To evaluate target engagement in skin biopsies.
To evaluate target engagement in hair follicles.
To evaluate target engagement in whole blood.
To explore additional exploratory biomarkers in blood samples.
To assess the effect of AZD5055 on biomarkers of bone turnover in serum samples, following oral administration of multiple ascending doses to healthy subjects.
To evaluate cardiac safety as assessed by 12-lead safety ECGs and dECGs, including the intention of obtaining a TQT substitute.
To evaluate changes in a panel of renal biomarkers in urine if indicated by the 3-month preclinical toxicity study.
To investigate the presence and measure levels of metabolites of AZD5055 in plasma and urine.
To assess the effect of AZD5055 on CYP3A (4-β-hydroxy-cholesterol).

A clinical trial according to the above protocol was carried out and can be summarised as follows:

### Main Inclusion Criteria

The study was conducted in healthy male and healthy female subjects of non-childbearing potential with suitable veins for cannulation or repeated venipuncture. The subjects had a body mass index between 18 and 30 kg/m² (inclusive) and weighed at least 50 kg. Also, the subjects were aged 18 to 55 years inclusive for men and women in Part 1 and only men in Part 2. For female subjects in Part 2, the age range was 18 to 49 years inclusive.

In Part 1 of the study, 3 dose levels of AZD5055 were investigated in 3 cohorts. Each cohort included 8 subjects. Within each cohort, 6 subjects were randomized to receive AZD5055, and 2 subjects were randomized to receive placebo. In Part 2 of the study, 3 dose levels of AZD5055 were investigated in 3 cohorts. In each cohort, 9 subjects were randomized to receive AZD5055, and 3 subjects were randomized to receive placebo. Naive subjects i.e. those who did not participate in Part 1 of this study, were included in Part 2 of the study. One subject in Cohort 1 who was randomized to receive AZD5055, did not receive AZD5055 treatment and withdrew from the study due to headache.

### Criteria for Evaluation

### Safety Variables

Safety endpoints included adverse events; vital signs (supine blood pressure, pulse, respiratory rate, and body temperature); 12-lead ECG, 12-lead dECG, telemetry, physical examinations, clinical laboratory assessments (hematology, clinical chemistry [including serum creatinine and creatine phosphokinase], urinalysis [including protein and albumin creatinine ratios], and cardiac biomarkers [including cardiac Troponin T (cTNT), cardiac Troponin I; (cTNI), and B type natriuretic peptide]), and peripheral oxygen saturation (SpO2).

### Pharmacokinetic Parameters

Primary PK parameters: Cₘₐₓ, AUC_{inf}, and AUCₗₐₛₜ; secondary PK parameters: including but not limited to tmax, AUC (0-12) or AUC (0-24) (the one not being the primary parameter), t_{1/2}λz, MRTinf, CL/F, Vz/F, Rac, TCP, Ae(t1-t2), fe (t1-t2), and CLR.

### Pharmacodynamic Parameters

Change from baseline in 4-β-hydroxy-cholesterol levels in blood.

### Statistical Methods

### Determination of Sample Size

The sample sizes for Part 1 and Part 2 of the study were chosen to obtain an adequate assessment of safety and tolerability without exposing undue numbers of subjects to the compound at this phase of clinical development. Previous experience in Phase I studies showed that the sample sizes proposed for Part 1 and Part 2 of the study were adequate to accomplish the objectives of the study. To enable an adequate analysis of the PK and safety of AZD5055, 8 evaluable subjects in the AZD5055 treatment arm were selected in Part 2 (MAD) Cohorts 1, 2 and 3. Eight evaluable subjects in the placebo arm were selected in Part 2 (MAD) Cohort.

### Presentation and Analysis of Safety Data

All safety data (scheduled and unscheduled) were presented in the data listings. Continuous variables were summarized using descriptive statistics (n, mean, standard deviation, min, median, max) by treatment. Categorical variables were summarized in frequency tables (frequency and proportion) by treatment (both Parts 1 and 2). The analysis of the safety variables was based on the safety analysis set. Adverse events were summarized by System Organ Class (SOC) and Preferred Term (PT) using Medical Dictionary for Regulatory Activities vocabulary. Furthermore, listings of serious adverse events (SAEs) and adverse events leading to the discontinuation of IMP (DAEs) were made and the number of subjects who had any adverse events (AEs), SAEs, DAEs and AEs with severe intensity were summarized. Adverse events that occur before dosing were reported separately. Tabulations and listings of data for ECG, vital signs, AEs, and clinical laboratory tests, measurements were presented.

### Presentation and Analysis of Pharmacokinetic Data

No formal statistical hypothesis testing was performed. The analyses of safety, tolerability, PK, and PD data was summarized descriptively by treatment/dose/study day separately using appropriate descriptive statistics including tables, listings, and graphs, as appropriate. Preliminary dose proportionality of AZD5055 after single dose (Day 1) in Part 1 and 2 and multiple dose (Day 16) in Part 2 was assessed graphically and was analyzed using the power model approach with the logarithm of PK parameters (AUC and Cₘₐₓ on Day 1) and (AUC(0-τ) and Cₘₐₓ on Day 16) as the dependent variable and the logarithm of the dose as the independent variable. For data collected in Part 2, the time dependency of the PK was evaluated by comparing AUC(0-τ) (Day 16) with AUC (Day 1) and accumulation was evaluated by comparing AUC(0-τ) (Day 16) with AUC(0-τ) (Day 1) and Cₘₐₓ (Day 16) with Cₘₐₓ (Day 1). A linear mixed-effect analysis of variance model using the logarithm of the above PK parameters as the response variable and treatment, day, and treatment by day interaction as fixed effects. Day was treated as a repeated effect within subject.

### Presentation and Analysis of Exploratory Data

Plasma 4-β-hydroxy-cholesterol levels at baseline and the last day of dosing were statistically analyzed using a linear mixed model analysis of covariance with treatment as a fixed effect and the baseline concentration of 4-β-hydroxy-cholesterol as covariate.

### Protocol Deviations

A total of 11 important protocol deviations were reported in 8 subjects. In Part of the study, 2 subjects had missing PK urine measurements, 1 subject had vomited within 2 hours of AZD5055 administration, and 1 subject had another protocol deviation related to urine sample collection. From these subjects, one subject was excluded from the PK analysis set (descriptive and inferential statistics) due to vomiting within 2 hours of AZD5055 administration (at/before 2 times median tmax) In Part 2 of the study, 4 subjects had time window deviations for PK plasma processing, 2 subjects had missing dECG measurements, and 1 subject had a time window deviation for PK urine processing reported. One subject was excluded from the PK analysis set as no PK sample was available, and one subject was excluded from the PD set as no PD sample was available. None of the reported deviations from the protocol influenced the interpretation of the study results.

### Results

### Pharmacokinetics

AZD5055 was rapidly absorbed following dosing with oral suspension of single ascending doses of 7 to 40 mg AZD5055. AZD5055 exposure increased approximately proportionally over the tested dose range of 5 to 40 mg after single and multiple dose administrations. A similar range of medians and a similar range of tmax of 1.00 to 1.30 hours was observed after multiple daily doses of 5 to 20 mg AZD5055. The AZD5055 concentrations declined in a bi-phasic manner with geometric mean terminal half-life values ranging 9.7 to 11.4 hours after single administration and ranging 12.2 to 17.2 hours after multiple daily administration of AZD5055. Between subjects, variability in the peak and extent of exposure (Cₘₐₓ and AUCs) was low (<25%) to moderate (>25%, <40%) respectively. The temporal change in systemic exposure was expected to be minimal. Little to moderate accumulation of AZD5055 was observed following repeat daily administration. Renal clearance of AZD5055 accounted for a small share of AZD5055 total clearance. Less than 2.3% of the dose administered was recovered unchanged (as AZD5055) in urine.

### Pharmacodynamics

There was no effect on 4-β-hydroxy-cholesterol following 14 days of once daily AZD5055 dosing, suggesting AZD5055 has no significant effect on CYP3A4 activity in healthy subjects.

### Safety

In Part 1, AZD5055 oral doses of 7, 20, and 40 mg (n=6 per dose level) administered as a single dose oral suspension were well-tolerated. In Part 2, AZD5055 was dosed at 5 mg QD, 15 mg QD and 20 mg QD as a single dose oral suspension, and was well tolerated at all administered doses. These assessments were based on the following:

### Part 1

There were no SAEs, deaths, or AEs leading to discontinuation of AZD5055 or withdrawal from the study.
Overall, 4 (22.2%) subjects across all AZD5055 doses and 1 (16.7%) subject in the pooled placebo group experienced at least one AE.
There were no notable trends for AEs by system organ class (SOC) or preferred term (PT). All AEs across all AZD5055 doses were reported only once.
Overall, 3 (16.7%) subjects across all AZD5055 doses and none in the pooled placebo group had at least one AE considered possibly related to AZD5055 as assessed by the Investigator. These AEs include dizziness, ventricular tachycardia, wheezing, upper abdominal pain, vomiting, and nausea.
All AEs were of Grade 1 (mild) intensity and recovered/resolved by the end of the study.
No clinically relevant trends were observed for clinical laboratory results, vital signs, physical examinations, and ECGs.

### Part 2

There were no SAEs or deaths.
There were 3 AEs leading to discontinuation of AZD5055.
   Two subjects who received the 5 mg AZD5055 dose experienced an AE of ventricular tachycardia/non-sustained ventricular tachycardia of Grade 2 (moderate) intensity, which were both considered to be possibly related to AZD5055 as assessed by the Investigator.
   One subject who received the 15 mg AZD5055 dose experienced an AE of coronavirus infection of Grade 1 (mild) intensity, which was not considered to be possibly related to AZD5055 as assessed by the Investigator.
Overall, 21 subjects across all AZD5055 doses and 5 subjects in the pooled placebo group experienced at least one AE.
There were no notable trends for A4s by system organ class (SOC) or preferred term (PT).
The majority of the AEs 16 were of Grade 1 (mild) intensity and there were 5 subjects (across all AZD5055 doses) with an AE of Grade 2 (moderate) intensity. All AEs recovered/resolved by the end of the study.
A total of 7 AEs across all AZD5055 doses and none in pooled placebo group were considered possibly related to AZD5055 treatment as assessed by the Investigator. These AEs included dysgeusia (1), headache (3), photophobia (1), ventricular tachycardia (2), and breath odour (1).
No clinically relevant trends were observed for clinical laboratory results, vital signs, physical examinations, and ECGs.

### Example 2. Second Clinical Study to Investigate Safety, Tolerability and Pharmacokinetics

The study comprises a screening period of maximum 28 days, and five periods during which subjects will participate from Day −1 of Period 1 to 72 hours after the AZD5055 dose in Period 5.

Period 1: On Day 1, the subjects will receive either 5 mg AZD5055 as a 20-minute IV infusion (Treatment A) in an overnight fasted state or AZD5055 20 mg as oral suspension (Treatment B) in an overnight fasted state.

Period 2: On Day 1 (Study Day 4), the subjects will receive 20 mg AZD5055 film-coated tablet (Treatment C) in an overnight fasted state.

Period 3: On Day 1 (Study Day 8) the subjects will receive a standardized high-fat breakfast 30 minutes before 20 mg AZD5055 administered as film-coated tablet (Treatment D).

Period 4: On Study Day 10, three days prior to Day 1, 20 mg rabeprazole will be administered twice daily. On Day 1 (Study Day 13), 20 mg AZD5055 film-coated tablet will be administered together with rabeprazole and rabeprazole dosing will continue twice daily (Treatment E).

Period 5: On Day 1 (Study Day 17) the participants will receive a low-fat breakfast 30 minutes before 20 mg AZD5055 film-coated tablet administered together with 20 mg rabeprazole.

Rabeprazole will continue twice daily, the last dose is on the evening of Study Day 18 (Treatment F).

A Follow-up Visit, or telephone call, approximately 6 days after the last AZD5055 dose in Period 5.

There will be a minimum washout of 3 days between the AZD5055 dose administration in Period 1 and Period 2 and a minimum washout of 4 days between AZD5055 doses administrations for subsequent study periods. Repeated PK (pharmacokinetic) sampling will be performed pre-dose to 48 hours after dosing of AZD5055 of each period. Subjects will be discharged on Study Day 20 (Day 4 of Period 5), after all the samples have been collected and all the assessments have been performed.

For single oral dose administration, 20 mg of AZD5055 was selected as once daily dosing of approximately 20 mg is predicted to achieve a C_{trough} (the trough concentration) of 23 nmol/L, which provides 24-hour coverage of 3-fold the in vitro IC₅₀ potency assay. This exposure is predicted to result in continuous inhibition of PORCN>75% in magnitude in the dosing interval. AZD5055 oral doses of 7, 20, and 40 mg (n=6 per dose level) administered as single dose oral suspension were well-tolerated in example 1.

The predicted human intestinal permeability is high with a fraction absorbed estimated to be near 100% and clearance is predicted to be low with limited first pass effect. Based on in vitro data, the predicted human oral bioavailability is 70%. The observed oral bioavailability in several animal species is in the range 40% to 60%. Based on these data, a lower bound on a plausible value for the oral bioavailability (F) for AZD5055 F was chosen as 25% or 0.25.

Based on the observed tmax (time to Cₘₐₓ) of 1 hour following an oral dose, it is estimated that approximately 75% of the dose is absorbed within 20 minutes. Therefore, a conservative estimate is that Cₘₐₓ following an IV infusion over 20 minutes is less than 2-fold higher compared to an oral dose, after correcting for F.

Rabeprazole is administered as oral doses of 20 mg BID in the morning and evening. Pre-treatment with rabeprazole for 3 days is required to reach the PD (pharmacodynamic) steady state of pH elevation prior to administration of AZD5055. The highest commonly recommended dose of 20 mg BID has not raised any notable safety concerns in preclinical and clinical studies

### Breakfasts

A high-fat, high-calorie breakfast constitutes the following: 2 eggs fried in butter; 2 strips of bacon; 2 slices of toast with butter; 112 g/4 oz of hash-brown potatoes; 240 mL/8 oz of whole milk. Substitutions in this test meal can be made as long as the meal provides a similar amount of calories from protein, carbohydrate, and fat and has comparable meal volume and viscosity.

A low-fat, low-calorie breakfast constitutes the following: 1 boiled egg; 1 packet flavoured instant oatmeal made with water; 240 mL/8 oz milk (1% fat). Substitutions in this test meal can be made as long as the meal provides a similar amount of calories from fat and has comparable meal volume and viscosity.

### IV Infusion

| | Quantity | | |
|---|---|---|---|
| Components | (per mL) | | Function |
| AZD5055 | 0.50 | mg | Active drug substance |
| Betadex sulfobutyl ether sodium | 60 | mg | Solubilising agent |
| Anhydrous citric acid | 0.55 | mg | Buffering agent |
| Sodium chloride | 4.0 | mg | Tonicity agent |

| | | |
|---|---|---|
| Hydrochloric acid | qs to pH 5 | pH adjusting agent |
| Sodium hydroxide | qs to pH 5 | pH adjusting agent |
| Water for injection | To 1.0 mL | Solvent |

### Primary Outcome Measures

Area under concentration time curve from time 0 to infinity (AUC_{inf}) [Time Frame: Day 1-6, 8-10, 13-15, 17-19]
Area under the concentration time curve from time 0 to the last quantifiable concentration (AUCₗₐₛₜ) [Time Frame: Day 1-6, 8-10, 13-15, 17-19]
Maximum observed concentration (Cₘₐₓ) [Time Frame: Day 1-6, 8-10, 13-15, 17-19]
   To estimate the relative bioavailability of AZD5055 film-coated tablet formulation versus AZD5055 oral suspension formulation.
   To estimate the absolute bioavailability of AZD5055 oral suspension and AZD5055 film-coated tablet formulation.
   To assess the effect of the proton pump inhibitor, rabeprazole, on the PK of AZD5055 alone and in combination with acid reducing agent.
   To assess the effect of the proton pump inhibitor, rabeprazole, on the PK of AZD5055, when AZD5055 is administered with food.

### Secondary Outcome Measures

In the time frame of Day 1-6, 8-10, 13-15, 17-19:
Time to reach maximum observed concentration (tmax)
Terminal elimination rate constant (λz)
Terminal elimination half-life (t_{1/2}λz)
Apparent total body clearance (extravascular dosing only) (CL/F)
Total body clearance (intravascular dosing only) (CL)
Apparent volume of distribution based on the terminal phase (extravascular dosing only) (Vz/F)
Volume of distribution based on the terminal phase (intravascular dosing only) (Vz)
Mean residence time (MRT)
Absolute bioavailability (Calculated based on geometric mean AUCinf of Treatment B or C vs Geometric mean AUCinf for Treatment A) (F)
Absolute bioavailability (Calculated based on geometric mean AUCinf of Treatment B or C vs Geometric mean AUCinf for Treatment A) (Fᵣₑₗ)
Ratio of Test Treatment to Reference Treatment based on AUCinf (Calculated for Treatment D vs Treatment C; Treatment E vs Treatment C; Treatment F vs Treatment C; Treatment F vs Treatment E) (R AUC))
Ratio of Test Treatment to Reference Treatment based on AUC_{inf} (Calculated for Treatment D vs Treatment C; Treatment E vs Treatment C; Treatment F vs Treatment C; Treatment F vs Treatment E) (R Cₘₐₓ))
   To estimate the relative bioavailability of AZD5055 film-coated tablet formulation versus AZD5055 oral suspension formulation.
   To estimate the absolute bioavailability of AZD5055 oral suspension and AZD5055 film-coated tablet formulation.
   To assess the effect of food on the pharmacokinetic (PK) parameters of AZD5055 in the fed and fasted state.
   To assess the effect of the acid reducing agent, rabeprazole, on the PK of AZD5055 alone and in combination with acid reducing agent.
   To assess the effect of the acid reducing agent, rabeprazole, on the PK of AZD5055, when AZD5055 is administered in fasted and fed state.
   To assess the safety following single oral and IV doses of AZD5055 in healthy participants.
Number of subjects with adverse events and serious adverse events [Time Frame: From screening (Day −28 to −2) to follow up (Day 23)]
   To assess the safety following single oral and IV doses of AZD5055 in healthy subjects

A clinical trial according to the above protocol was carried out and can be summarised as follows:

### Main Inclusion Criteria

Healthy male and female (of non-childbearing potential) participants aged 18 to 55 years, who had a body mass index (BMI) between 18 and 30 kg/m2, inclusive.

### Criteria for Evaluation

Pharmacokinetic Parameters—Pharmacokinetic parameters of AZD5055 included, but were not limited to, Cₘₐₓ, AUC_{inf}, and AUCₗₐₛₜ.

Safety Variables—Safety endpoints included adverse events (AEs), clinical laboratory assessments (hematology, clinical chemistry, urinalysis, and urinary albumin:creatinine ratios), vital signs and peripheral oxygen saturation (SpO2), standard 12-lead electrocardiogram, (ECG), telemetry, and physical examinations.

### Statistical Methods

### Determination of Sample Size

Approximately 18 healthy participants were enrolled to ensure that at least 15 participants completed the 5-period study. It was expected that the proposed sample size would give adequate information on the effect of formulation and concomitant treatment on the exposure of AZD5055, while exposing as few participants as possible to study procedures. Interpretation of the results were based on the estimated geometric mean ratio (GMR) and the associated 90% confidence interval (CI) between the combination of test and reference for AUC and Cₘₐₓ.

### Presentation and Analysis of Pharmacokinetic Data

Plasma concentrations were listed for each participant at each actual sampling time and summarized by treatment and nominal sampling time, using the same descriptive statistics as for the PK parameters. Graphical presentations included individual and geometric mean time-concentration curves (actual sampling time for individual curves and nominal sampling time for mean curves) on linear and semi-logarithmic scale. Pharmacokinetic variables and diagnostics of the PK analysis were listed, and PK variables were summarized by treatment using descriptive statistics (n, geometric mean, geometric coefficient of variation [gCV; %], arithmetic mean, arithmetic standard deviation [SD], minimum [min], median, and maximum [max]) for all variables except tmax, which were summarized using n, min, median, and max. For the assessment of absolute and relative bioavailability of AZD5055 the following statistical comparisons were made on the PK analysis set:
AZD5055 film-coated tablet, fasted, C (test)/AZD5055 oral suspension, fasted, B (reference).
AZD5055 film-coated tablet, fasted, C (test)/AZD5055 solution for infusion, A (reference).

The analyses were performed using a linear mixed-effects analysis of variance (ANOVA) model using the natural logarithm of Cₘₐₓ, AUC_{inf}, and AUCₗₐₛₜ as the response variables; sequence, period and treatment as fixed effect, and participant nested within sequence as random effect. Transformed back from the logarithmic scale, geometric means together with CIs (2-sided 95%) for Cₘₐₓ, AUC_{inf}, and AUCₗₐₛₜ were estimated and presented.

The result was transformed back to the original scale in order to give estimates of true */geometric standard deviation (gSD) ratios and 90% CIs for these ratios. Additionally, the 90% CI for the difference was calculated and presented.

For the assessment of food effect and effect of acid reducing agents the following statistical comparisons were made on the PK analysis set:
AZD5055 film-coated tablet 20 mg, fed state, D (test)/AZD5055 film-coated tablet 20 mg, overnight fasted state, C (reference).
AZD5055 film-coated tablet fed, with rabeprazole, F (test)/AZD5055 film-coated tablet overnight fasted, with rabeprazole, E (reference).
AZD5055 film-coated tablet overnight fasted, with rabeprazole, E (test)/AZD5055 film-coated tablet 20 mg, overnight fasted state, C (reference).
AZD5055 film-coated tablet fed, with rabeprazole, F (test)/AZD5055 film-coated tablet 20 mg, overnight fasted state, C (reference).

The effect of food and acid reducing agents on the PK of AZD5055 was assessed, using a linear mixed-effects ANOVA model with fixed effects for treatment, period, and sequence, and a random effect for participant within sequence. This analysis was performed using natural logarithmic transformed Cₘₐₓ, AUCₗₐₛₜ, and AUC_{inf}. Transformed back from the logarithmic scale, geometric means together with CIs (2-sided 95%) for Cₘₐₓ, AUCₗₐₛₜ, and AUC_{inf} were calculated and presented. Also, ratios of geometric means together with CIs (2-sided 90%) were estimated and presented for treatment comparison (ie, AZD5055 under fed condition versus AZD5055 under fasted conditions).

### Presentation and Analysis of Safety Data

All safety data (scheduled and unscheduled) were presented in the data listings. Continuous variables were summarized using descriptive statistics (n, mean, SD, min, median, max) by treatment. Categorical variables were summarized in frequency tables (frequency and proportion) by treatment/dose group. The analysis of the safety variables was based on the safety analysis set.

### Protocol Deviations

There were 2 participants with 2 important protocol deviations in Treatment Sequence ACDEF and 3 participants with 4 important protocol deviations in Treatment Sequence BCDEF. None of the important protocol deviations are assumed to affect any of the pre-specified endpoints. Therefore, no participant with an important protocol deviation was excluded from any analysis set.

### Results

### Pharmacokinetic Results

Absolute bioavailability of the film-coated tablet was approximately 66% and 64% based on AUC_{inf} and AUCₗₐₛₜ. Cₘₐₓ for the film-coated tablet was approximately 21% of the IV dose.
Total systemic exposure to AZD5055 following dosing with the film-coated tablet was comparable to dosing with the oral suspension based on AUC_{inf} and AUCₗₐₛₜ, however Cₘₐₓ was approximately 35% lower.
Exposure to AZD5055 based on Cₘₐₓ, AUCₗₐₛₜ and AUC_{inf} increased by between 22% and 32% when dosed in a fed state compared to fasted. However, when dosed with rabeprazole, food did not impact total systemic exposure to AZD5055, while it increased Cₘₐₓ by approximately 17% compared to dosing with rabeprazole fasted.
When dosed with rabeprazole in the fasted state there was no change in total systemic exposure (AUC_{inf} and AUCₗₐₛₜ) compared to AZD5055 fasted alone, however Cₘₐₓ was reduced by 32%.
When dosed with rabeprazole in the fed state there was no change in total systemic exposure (AUC_{inf} and AUCₗₐₛₜ) compared to AZD5055 fasted alone, however Cₘₐₓ was reduced by 20%.

### Safety Results

No major safety or tolerability concerns were identified in this study and AZD5055 was generally well tolerated. This assessment was based on the following:
There were no deaths in this study. One participant experienced an SAE in Period 3. This SAE was assessed by the Investigator to be possibly related to AZD5055 (transaminases increased).
Two participants experienced an AE (2 events) leading to discontinuation of AZD5055 after dosing in Period 3 (transaminases increased and non-cardiac chest pain).
Overall, 11 participants experienced any AE (20 events) during the study. Five participants experience any AE (9 events) assessed by the Investigator to be possibly related to AZD5055.
In total, 8 participants reported at least 1 AE of Grade 1 intensity, and 4 participants reported at least 1 AE of Grade 2 intensity. No AEs Grade 3 or higher were reported.
No clinically relevant trends were observed for laboratory parameters, vital signs, ECGs, or physical examination.
The coronavirus disease 2019 (COVID-19) pandemic did not impact the overall safety results of this study.

### REFERENCES

A number of publications are cited above. Full citations for these references are provided below. The entirety of each of these references is incorporated herein.

| Author | Reference | Doi |
|---|---|---|
| Adegunsoye | Adegunsoye A, et al., Chest. 2019 | 10.1016/j.chest.2018.12.011. |
| 2019 | May; 155(5): 1026-40 | |
| Baarsma and | Baarsma H A, Königshoff M. | 10.1136/thoraxjnl-2016-209753. |
| Königshoff 2017 | Thorax. 2017 August; 72(8): 746-59 | |
| Baumgartner | Baumgartner K B, et al., | 10.1164/ajrccm.155.1.9001319. |
| 1997 | Am J Respir Crit Care Med. 1997 | |
| | Janurary; 155(1): 242-8 | |
| Bunyard 2019 | Bunjard, J. et al., Eur Respir J | |
| | 2019; 54: Suppl. 63, 589 | |
| Chen 2017 | Chen C W, et al., | 10.1136/annrheumdis-2016- |
| | Ann Rheum Dis. 2017 April; 76(4): | 210294. |
| | 773-8. | |
| Chilosi 2003 | Chilosi M, et al., | 10.1016/s0002-9440(10)64282- |
| | Am J Pathol. 2003 May; 162(5): | 4. |
| | 1495-502 | |
| Distler 2019 | Distler J H W, et al., | 10.1038/s41584-019-0322-7 |
| | Nat Rev Rheumatol. 2019 December; | |
| | 15(12): 705-30 | |
| Herr 2012 | Herr P, Basler K. Dev Biol. 2012 | 10.1016/j.ydbio.2011.11.003 |
| | Jan. 15; 361(2): 392-402 | |
| Hutchinson | Hutchinson J, et al., | 10.1183/09031936.00185114 |
| 2015 | Eur Respir J. 2015 September; 46(3): | |
| | 795-806 | |
| King 2014 | King T E Jr, et al., | 10.1056/NEJMoa1402582 |
| | N Engl J Med. 2014 | |
| | May 29; 370(22): 2083-92 | |
| Khanna 2020 | Khanna D, et al., | 10.1016/S2213-2600(20)30318-0 |
| | Lancet Respir Med. | |
| | 2020 October; 8(10): 963-74. | |
| Königshoff 2009 | Königshoff M, et al., | 10.1172/JCI33950. |
| | J Clin Invest. 2009 April; 119(4): | |
| | 772-87 | |
| Königshoff 2008 | Königshoff M, | 10.1371/journal.pone.0002142 |
| | et al., PLoS One. 2008 May 14; | |
| | 3(5): e2142 | |
| Lam 2011 | Lam A P, et al., Am J Respir Cell | 10.1165/rcmb.2010- |
| | Mol Biol. 2011 November; 45(5): 915-22 | 0113OC |
| Lam 2014 | Lam A P, et al., | 10.1164/rccm.201401-0079OC |
| | Am J Respir Crit Care Med. 2014 | |
| | Jul. 15; 190(2): 185-95 | |
| Ley 2011 | Ley B, et al., | 10.1164/rccm.201006-0894CI |
| | Am J Respir Crit Care Med. 2011 | |
| | Feb. 15; 183(4): 431-40 | |
| Madan 2016 | Madan B, et al., | 10.1016/j.kint.2016.01.017 |
| | Kidney Int. 2016 May; 89(5): 1062- | |
| | 74 | |
| Oda 2016 | Oda K, et al., | 10.1186/s12931-016-0357-0 |
| | Respir Res. 2016 Apr. 12; | |
| | 17: 39 | |
| Richeldi 2017 | Richeldi L, et al., Lancet. 2017 May | 10.1016/S0140-6736(17)30866-8 |
| | 13; 389(10082): 1941-52 | |
| Richeldi 2014 | Richeldi L, et al., | 10.1056/NEJMoa1402584 |
| | N Engl J Med. 2014 May 29; | |
| | 370(22): 2071-82 | |
| Rodon 2021 | Rodon J, et al., | 10.1038/s41416-021-01389-8 |
| | Br J Cancer. 2021; 125(1): 28-37 | |
| Shah 2021 | Shah K, et al., | 10.1016/j.phrs.2021.105532 |
| | Pharmacol Res. 2021 May; 167: | |
| | 105532 | |
| Spanjer 2016 | Spanjer AIR, et al., | 10.1096/fj.201500129 |
| | FASEB J. 2016 May; 30(5): 1823- | |
| | 35 | |
| Wells 2020 | Wells AU, et al., | 10.1016/S2213- |
| | Lancet Respir Med. 2020 May; | 2600(20)30036-9 |
| | 8(5): 453-60 | |
| Wollin 2019 | Wollin L, et al., | 10.1183/13993003.00161-2019 |
| | Eur Respir J. 2019 Sep. 19; 54(3): | |
| | 1900161 | |
| Yu 2019 | Yu M, et al., | 10.1016/j.imbio.2019.04.006 |
| | Immunobiology. 2019 July; 224(4): | |
| | 551-9 | |

1. A method of treating interstitial lung disease in a subject in need thereof, comprising orally administering to the subject 2-[4-(2-methyl-4-pyridyl)pyrrolo[3,2-c]pyridin-1-yl]-N-(5-pyrazin-2-yl-2-pyridyl)acetamide (AZD5055): 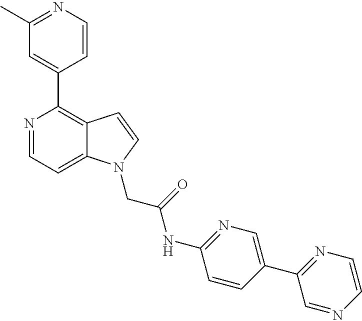
in a dose of between 5 and 35 mg per day.

2. The method according to claim 1, wherein the dose per day is between 10 and 30 mg per day.

3. The method according to either claim 1 or claim 2, wherein the dose per day is between 15 and 25 mg per day.

4. The method according to claim 1, wherein the dose per day is 5 mg, about 10 mg, about 15 mg, about 20 mg, about 25 mg, about 30 mg or 35 mg per day.

5. The method according to claim 1, wherein the dose per day is selected from the group consisting of: 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg and 35 mg.

6. The method according to claim 1, wherein the dose per day is about 5 mg per day.

7. The method according to claim 1, wherein the dose per day is about 15 mg per day.

8. The method according to any one of claim 1 to 7, wherein AZD5055 is administered once a day (QD).

9. The method according to any one of claim 1 to 7, wherein AZD5055 is administered twice a day (BID).

10. The method according to any one of claims 1 to 9, wherein AZD5055 is continuously administered in a treatment cycle.

11. The method according to any one of claims 1 to 10, wherein AZD5055 is taken on an empty stomach, with no food two hours before, and one hour after.

12. The method according to any one of claims 1 to 10, wherein AZD5055 is taken with food.

13. The method according to any one of claims 1 to 12, which additionally comprises the separate, sequential or simultaneous administration of a proton pump inhibitor or a pharmaceutically acceptable salt thereof.

14. The method according to claim 13, wherein the proton pump inhibitor is rabeprazole.

15. The method according to claim 14, wherein rabeprazole is administered in a dose of about 10 or 20 mg orally twice a day (BID).

16. The method according to any one of claims 1 to 15, wherein the interstitial lung disease is idiopathic pulmonary fibrosis.

17. An oral dosage unit which comprises 2-[4-(2-methyl-4-pyridyl)pyrrolo[3,2-c]pyridin-1-yl]-N-(5-pyrazin-2-yl-2-pyridyl)acetamide (AZD5055): 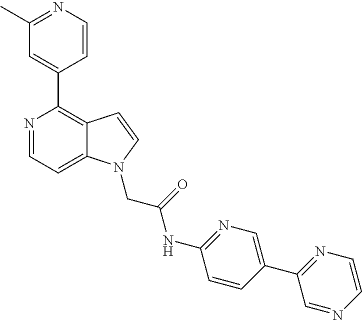
in an amount of between 2.5 and 35 mg.

18. The oral dosage unit according to claim 17, which comprises the daily dose of AZD5055 as defined in any of claims 1 to 7.

19. The oral dosage unit according to claim 17, which comprises half the daily dose of AZD5055 as defined in any of claims 1 to 7.

20. The oral dosage unit according to any one of claims 17 to 19, wherein the oral dosage unit is an aqueous suspension.

21. The oral dosage unit according to claim 20, wherein the suspension comprises one or more additional excipients selected from the group consisting of: viscosity agents, sweeteners and colorants.

22. The oral dosage unit according to any one of claims 17 to 19, wherein the oral dosage unit is a tablet.

23. The oral dosage unit according to claim 22, wherein the tablet comprises one or more pharmaceutical excipients selected from the group consisting of: diluents, compression aids, disintegrants, and glidants/lubricants.

24. The oral dosage unit according to claim 23, wherein a diluent is present, and the diluent is a sugar or sugar-containing, except mannitol, or an inorganic compound.

25. The oral dosage unit according to claim 24, wherein, the diluent is microcrystalline cellulose.

26. The oral dosage unit according to any one of claims 23 to 25, wherein a compression aid is present, and is selected from microcrystalline cellulose (MCC), xylitol, di-calcium phosphate, lactose monohydrate and starch.

27. The oral dosage unit according to claim 26, wherein the compression aid is di-calcium phosphate.

28. The oral dosage unit according to either claim 26 or claim 27, wherein the compression aid is between 10 and 30 wt % of the tablet core.

29. The oral dosage unit according to any one of claims 23 to 28, wherein a lubricant/glidant is present and is glyceryl behenate.

30. The oral dosage unit according to claim 29, wherein the lubricant/glidant is between 2 and 15 wt % of the tablet core.

31. The oral dosage unit according to any one of claims 23 to 30, wherein a disintegrant is present, and is a compound which swells or dissolves in water.

32. The oral dosage unit according to claim 31, wherein the disintegrant is croscarmellose sodium.

33. The oral dosage unit according to either claim 31 or claim 32, wherein the disintegrant is between 2 and 15 wt % of the tablet core.

34. The oral dosage unit according to any one of claims 23 to 33, wherein the tablet is coated.
